(19) United States
(12) Patent Application Publication
(10) Pub. No.: US 2011/0045405 A1
(43) Pub. Date: Feb. 24, 2011

(54) (Meth)acrylate compound, photosensitive polymer, and resist composition

(76) Inventors: Sung-Jae Lee, Uiwang-si (KR); Tae-Ho Kim, Uiwang-si (KR); Jun-Suk Kim, Uiwang-si (KR); Jin-Young Lee, Uiwang-si (KR); Kang Ryu, Uiwang-si (KR); Sang-Jun Choi, Uiwang-si (KR)

Correspondence Address: LEE & MORSE, P.C., 3141 FAIRVIEW PARK DRIVE, SUITE 500, FALLS CHURCH, VA, 22042, US

(21) Appl. No.: 12/805,726

(22) Filed: Aug. 17, 2010

(30) Foreign Application Priority Data
Aug. 21, 2009 (KR) 10-2009-0077746

(51) Int. Cl. G03F 7/004; C07D 307/93; C07D 495/04; C08F 24/00; C08F 28/06

(52) U.S. Cl. 430/270.1; 549/300; 549/45; 526/268; 526/270; 526/256

(57) ABSTRACT

Disclosed are a (meth)acrylate compound, a photosensitive polymer, and a resist composition, and the (meth)acrylate compound includes a lactone-group-containing (meth)acrylate compound represented by the following Chemical Formula 1.

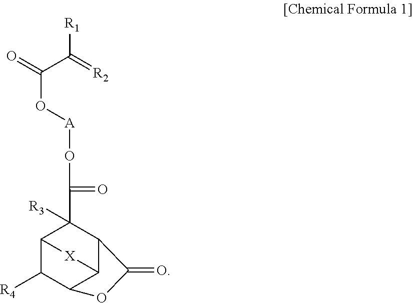

### BACKGROUND

1. Field

Embodiments relate to a (meth)acrylate compound, a photosensitive polymer, and a resist composition.

2. Description of the Related Art

Complications of a semiconductor manufacturing process and integration of semiconductors have increasingly required forming a fine pattern. For example, a semiconductor device with a capacity of more than 16 gigabytes may have a pattern size of less than 70 nm according to a design rule. Lithographic formation of such patterns may require a thin resist film.

For lithography, a photoresist material using a shorter wavelength, such as an ArF excimer laser of 193 nm, is preferred to one using a longer wavelength, such as a KrF excimer laser of 248 nm. However, a process margin at underlayer etching has been reduced, and thus a general resist material for use with an ArF excimer laser may be approaching a limit.

### SUMMARY

It is a feature of an embodiment to provide a lactone-group-containing (meth)acrylate compound having a low manufacturing cost and suitable for preparation of a photosensitive polymer due to improved sensitivity, resolution, and etching resistance of the monomer.

It is another feature of an embodiment to provide a photosensitive polymer including a repeating unit derived from the (meth)acrylate compound and having excellent sensitivity, resolution, and etching resistance.

It is another feature of an embodiment to provide a resist composition including the photosensitive polymer and providing excellent lithography performance in a lithographic process using a light source emitting light in an ultrashort wavelength region such as a 193 nm region or EUV (13.5 nm).

At least one of the above and other features and advantages may be realized by providing a lactone-group-containing (meth)acrylate compound represented by the following Chemical Formula 1,

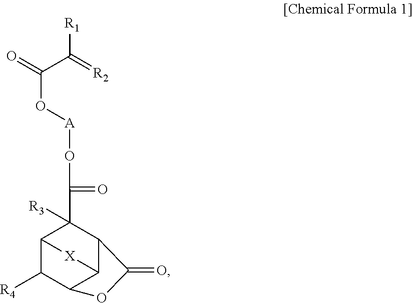

wherein, in the above Chemical Formula 1, R₁ is hydrogen, a methyl group, or CH₂CO₂R₅,

R₂ is CR′R″ where R′ and R″ are independently hydrogen, a methyl group, or CO₂R₅,

R₃ is hydrogen, or a linear, branched, or cyclic substituted or unsubstituted alkyl group,

R₄ is a hydroxy or CO₂R₅,

X is (CH₂)ₙ (where n is 1 or 2), O, or S,

R₅ is a linear, branched, or cyclic substituted or unsubstituted alkyl group, and

A is a linear, branched, or cyclic substituted or unsubstituted alkylene group.

The (meth)acrylate compound may be a compound represented by one of the following Chemical Formulae 1a to 1p,

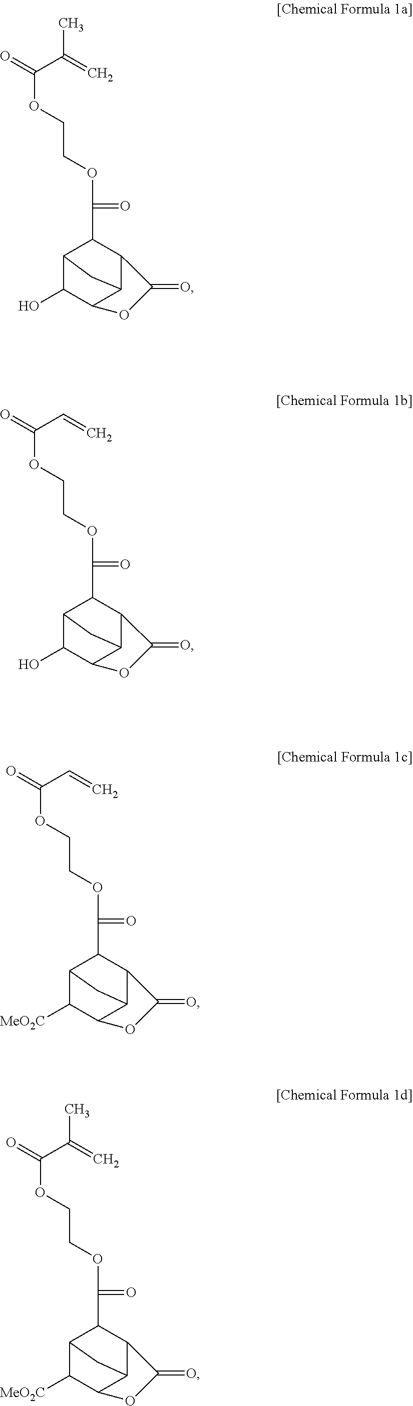

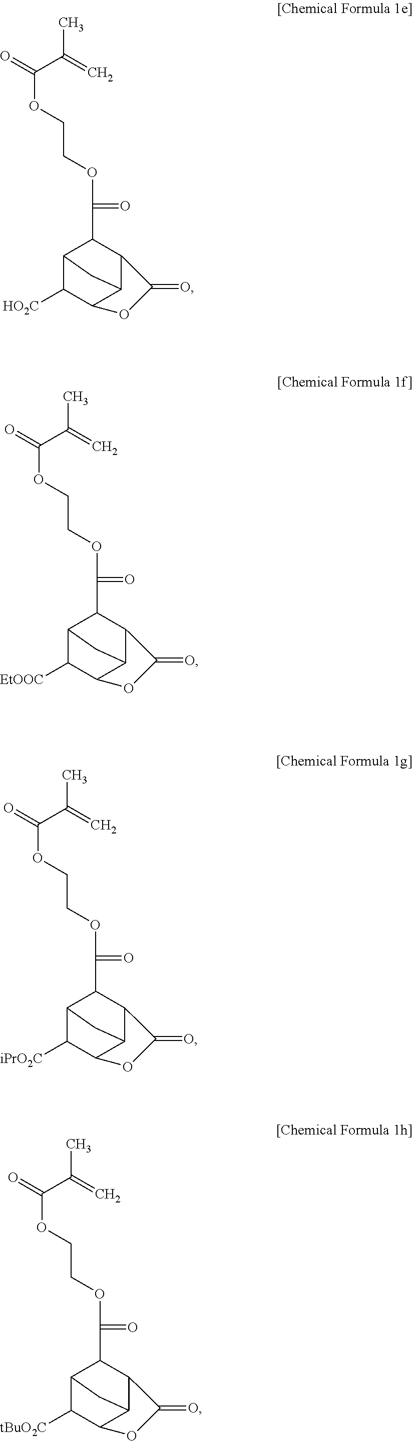

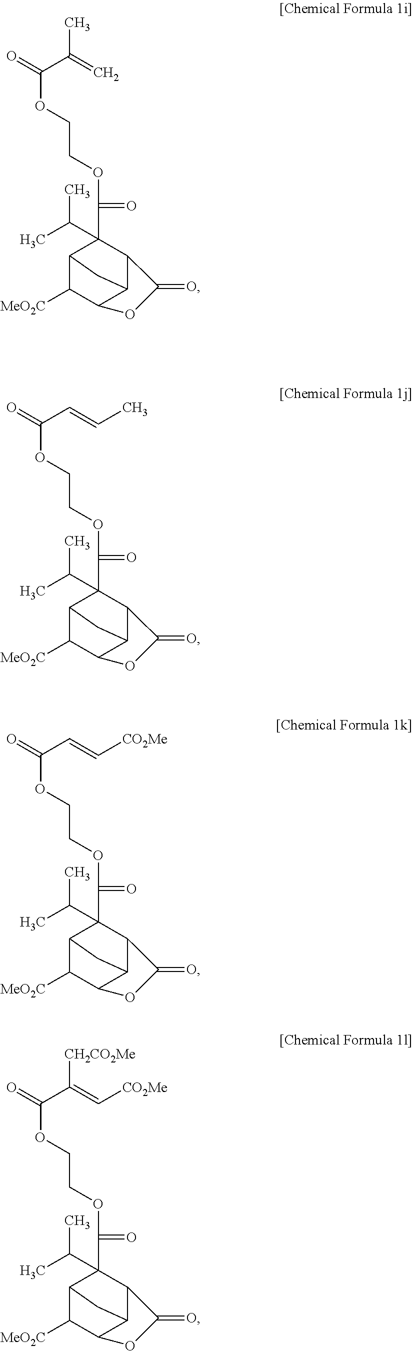

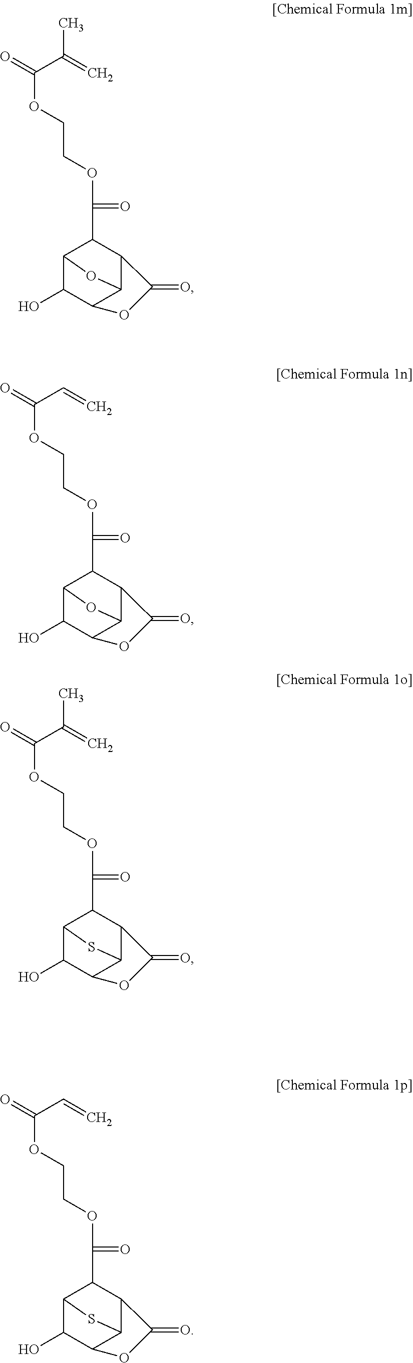

At least one of the above and other features and advantages may also be realized by providing a photosensitive polymer, including a repeating unit derived from a compound represented by the following Chemical Formula 1, and a repeating unit derived from a compound represented by the following Chemical Formula 2,

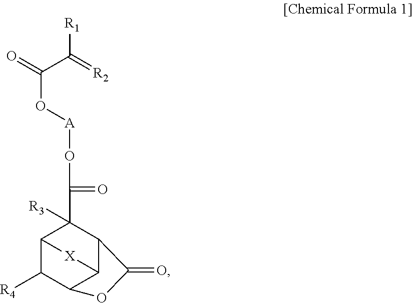

wherein, in the above Chemical Formula 1, R₁ is hydrogen, a methyl group, or CH₂CO₂R₅,

R₂ is CR′R″ where R′ and R″ are independently hydrogen, a methyl group, or CO₂R₅,

R₃ is hydrogen, or a linear, branched, or cyclic substituted or unsubstituted alkyl group,

R₄ is a hydroxy or CO₂R₅,

R₅ is a linear, branched, or cyclic substituted or unsubstituted alkyl group,

X is (CH₂)ₙ (where n is 1 or 2), O, or S, and

A is a linear, branched, or cyclic substituted or unsubstituted alkylene group,

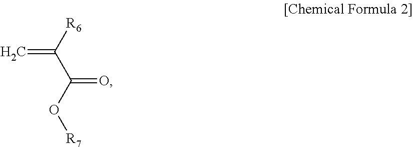

wherein, in the above Chemical Formula 2, R₆ is hydrogen or a methyl group, and

R₇ is a C₄ to C₂₀ acid-labile group being decomposed under an acid catalyst.

R₇ may be an alkyl group, a norbornyl group, an isobornyl group, a cyclodecanyl group, a norbornyl having a lower alkyl substituent, an isobornyl having a lower alkyl substituent, a cyclodecanyl group having a lower alkyl substituent, an adamantyl group having a lower alkyl substituent, alkoxycarbonyl, alkoxycarbonyl alkyl, amyloxycarbonyl, amyloxycarbonyl alkyl, 2-tetrahydropyranyloxycarbonyl alkyl, 2-tetrahydrofuranyloxycarbonyl alkyl, a tertiary alkyl group, or an acetal group.

A mole fraction of the repeating unit derived from the compound represented by Chemical Formula 1 may range from 0.2 to 0.8 and a mole fraction of the repeating unit derived from the compound represented by Chemical Formula 2 may range from 0.2 to 0.8 based on the total moles of the repeating units derived from the compounds represented by Chemical Formulae 1 and 2.

The photosensitive polymer may further include a repeating unit derived from a compound represented by the following Chemical Formula 3,

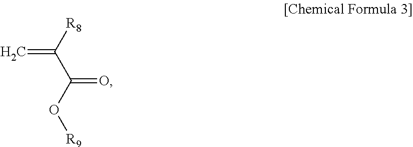

wherein, in the above Chemical Formula 3, R₈ is hydrogen or a methyl group, and

R₉ is hydrogen or an alkyl group or cycloalkyl group including a polar functional group of a hydroxy group, a carboxyl group, or a combination thereof.

R₉ may be 2-hydroxyethyl or 3-hydroxy-1-adamantyl.

The photosensitive polymer may further include a repeating unit derived from a compound represented by the following Chemical Formula 4,

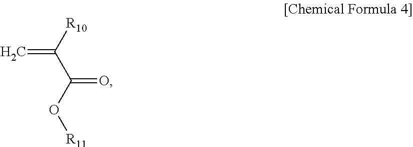

wherein, in the above Chemical Formula 4, R₁₀ is hydrogen or a methyl group, and

R₁₁ is a lactone-derived group.

R₁₁ may be a substituent represented by the following Chemical Formula 5 or Chemical Formula 6,

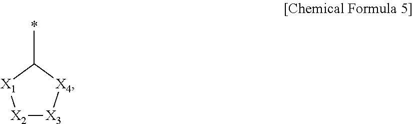

wherein, in the above Chemical Formula 5, two of X₁ to X₄ positioned to be adjacent to each other are CO and O, and the remaining two of X₁ to X₄ except for CO and O are CR″ (where R″ is hydrogen, a C₁ to C₄ alkyl, or an alkylene forming a fused ring with a five-member ring),

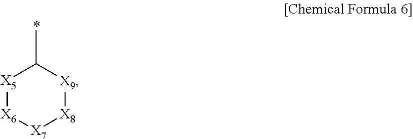

wherein, in the above Chemical Formula 6, two of X₅ to X₉ positioned to be adjacent to each other are CO and O, and the remaining three of X₅ to X₉ except for CO and O are CR″ (where R″ is hydrogen, a C₁ to C₄ alkyl, or an alkylene forming a fused ring with a six-member ring); or X₅ to X₉ are CR′″ (where R′″ is hydrogen, a C₁ to C₄ alkyl, an ester-containing alkylene forming a fused ring with the six-member ring), and at least two of R′″ are linked to each other to from a lactone ring.

The photosensitive polymer may have a weight average molecular weight (Mw) of about 3,000 to about 20,000.

The photosensitive polymer may have a polydispersity (Mw/Mn) of about 1.5 to about 2.5.

At least one of the above and other features and advantages may also be realized by providing a resist composition, including a photosensitive polymer including a repeating unit derived from a compound represented by the following Chemical Formula 1 and a repeating unit derived from a compound represented by the following Chemical Formula 2, a photoacid generator (PAG), and a solvent,

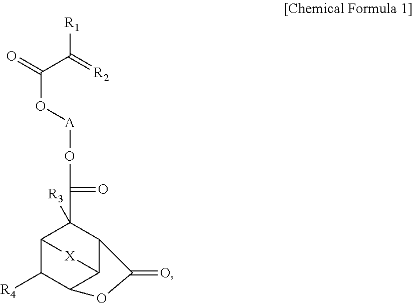

wherein, in the above Chemical Formula 1, R₁ is hydrogen, a methyl group, or CH₂CO₂R₅,

R₂ is CR′R″ where R′ and R″ are independently hydrogen, a methyl group, or CO₂R₅,

R₃ is hydrogen, or a linear, branched, or cyclic substituted or unsubstituted alkyl group,

R₄ is a hydroxy or CO₂R₅,

R₅ is a linear, branched, or cyclic substituted or unsubstituted alkyl group,

X is (CH₂)ₙ (where n is 1 or 2), O, or S, and

A is a linear, branched, or cyclic substituted or unsubstituted alkylene group,

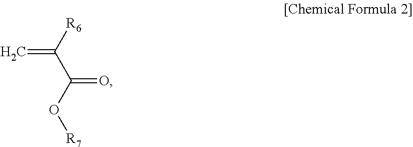

wherein, in the above Chemical Formula 2, R₆ is hydrogen or a methyl group, and

R₇ is a C₄ to C₂₀ acid-labile group being decomposed under an acid catalyst.

The photosensitive polymer may further include a repeating unit derived from a compound represented by the following Chemical Formula 3,

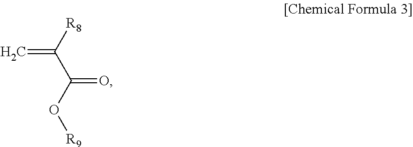

wherein, in the above Chemical Formula 3, R₈ is hydrogen or a methyl group, and

R₉ is hydrogen, an alkyl group, or a cycloalkyl group, the alkyl group and the cycloalkyl group including a hydroxy group, a carboxyl group, or a combination thereof.

The photosensitive polymer may further include a repeating unit derived from a compound represented by the following Chemical Formula 4,

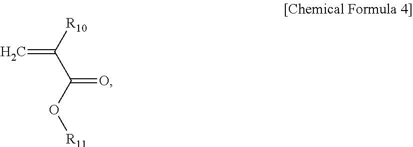

wherein, in the above Chemical Formula 4, R₁₀ is hydrogen or a methyl group, and

R₁₁ is a lactone-derived group.

The photosensitive polymer may be included in an amount of about 5 to about 15 parts by weight based on 100 parts by weight of a resist composition.

The photoacid generator may be added in an amount of about 1 to about 15 parts by weight based on 100 parts by weight of the photosensitive polymer.

The photoacid generator may include a triarylsulfonium salt, a diaryl iodonium salt, a sulfonate, or a mixture thereof.

The organic base may be included in an amount of about 0.1 to about 1 part by weight based on 100 parts by weight of the photosensitive polymer.

The organic base may include triethylamine, triisobutylamine, trioctylamine, triisodecylamine, triethanolamine, or a mixture thereof.

### BRIEF DESCRIPTION OF THE DRAWING

The above and other features and advantages will become more apparent to those of skill in the art by describing in detail example embodiments with reference to the attached drawing, in which:

FIG. 1 illustrates a graph showing NMR of a monomer according to Example 1-1.

### DETAILED DESCRIPTION

Korean Patent Application No. 10-2009-0077746, filed on Aug. 21, 2009, in the Korean Intellectual Property Office, and entitled: “(Meth)Acrylate Compound, Photosensitive Polymer, and Resist Composition,” is incorporated by reference herein in its entirety.

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey the scope of the invention to those skilled in the art.

As used herein, when specific definition is not otherwise provided, the term “alkyl” refers to a C₁ to C₈ alkyl, the term “lower alkyl” refers to a C₁ to C₄ alkyl, the term “alkoxy” refers to C₁ to C₂₀ alkoxy, for example a C₁ to C₁₂ alkoxy, the term “alkylene” refers to a C₁ to C₂₀ alkylene, for example a C₁ to C₁₂ alkylene, the term “cycloalkyl” refers to C₃ to C₁₄ cycloalkyl, and the term “aryl” refers to C₆ to C₂₀ aryl.

In the present specification, the term “substituted” refers to a functional group substituted with an alkyl group or an aryl group instead of at least one of hydrogen.

In an embodiment, a novel lactone-group-containing (meth)acrylate compound is represented by the following Chemical Formula 1.

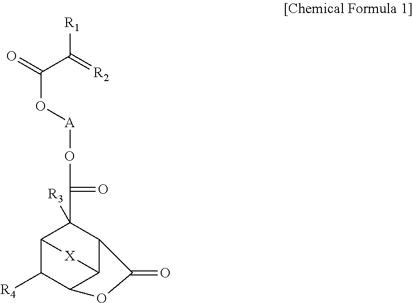

wherein, R₁ is hydrogen, a methyl group, or CH₂CO₂R₅,

R₂ is CR′R″ where R′ and R″ are independently hydrogen, a methyl group, or CO₂R₅,

R₃ is hydrogen, or a linear, branched, or cyclic substituted or unsubstituted alkyl group, for example a methyl group, an ethyl group, a propyl group, an isopropyl group, an n-butyl group, a sec-butyl group, a tert-butyl group, a tert-amyl group, an n-pentyl group, an n-hexyl group, a cyclopentyl group, a cyclohexyl group, a cyclopentylmethyl group, a cyclopentylethyl group, a cyclohexylmethyl group, or cyclohexylethyl group, and the like,

R₄ is a hydroxy or CO₂R₅,

R₅ is a linear, branched, or cyclic substituted or unsubstituted alkyl group, for example a methyl group, an ethyl group, a propyl group, an isopropyl group, an n-butyl group, a sec-butyl group, a tert-butyl group, a tert-amyl group, an n-pentyl group, an n-hexyl group, a cyclopentyl group, a cyclohexyl group, an ethylcyclopentyl group, a butylcyclopentyl group, an ethylcyclohexyl group, a butylcyclohexyl group, an adamantyl group, an ethyl adamantyl group, a butyl adamantyl group, and the like,

X is (CH₂)ₙ (where n is 1 or 2), O, or S, and

A is a linear, branched, or cyclic substituted or unsubstituted alkylene group, for example a C₁ to C₆ alkylene group.

Examples of the (meth)acrylate compound having the above Chemical Formula 1 include compounds represented by the following Chemical Formulae 1a to 1p, but are not limited thereto.

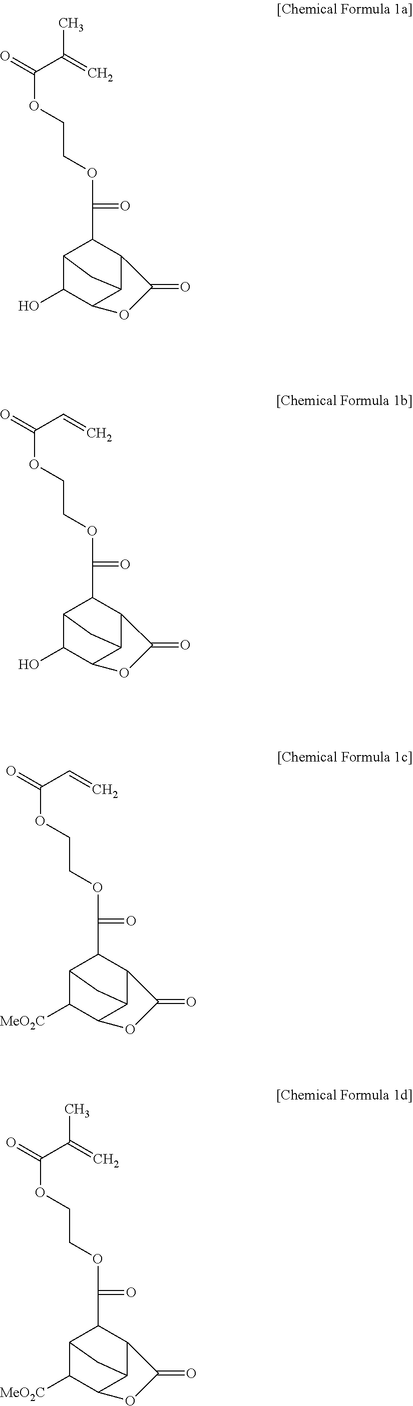

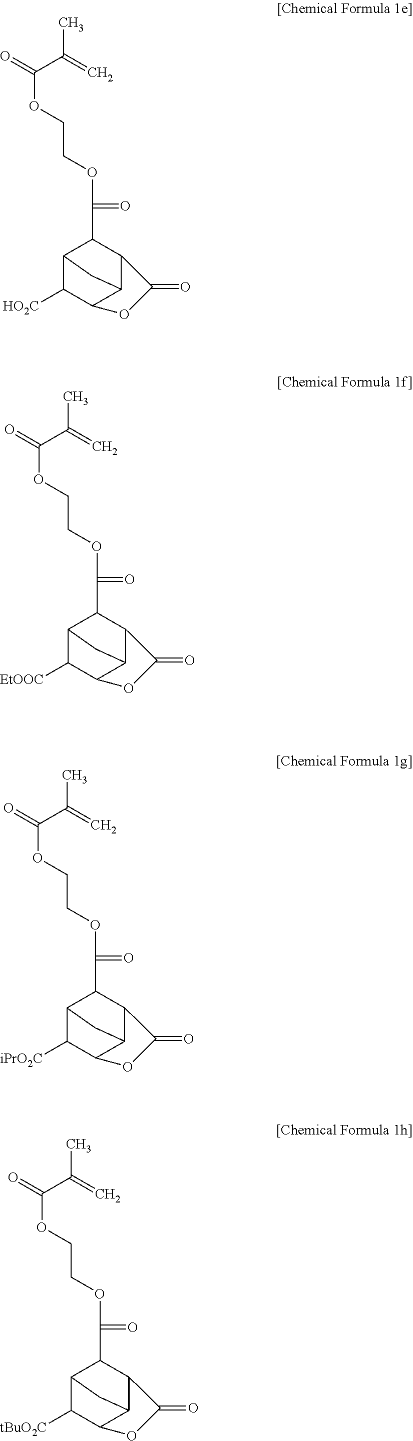

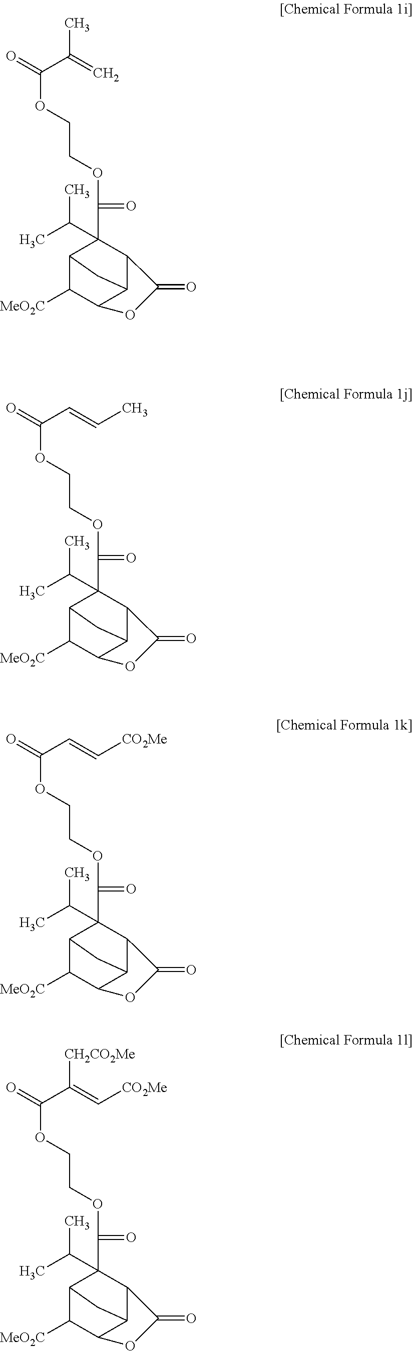

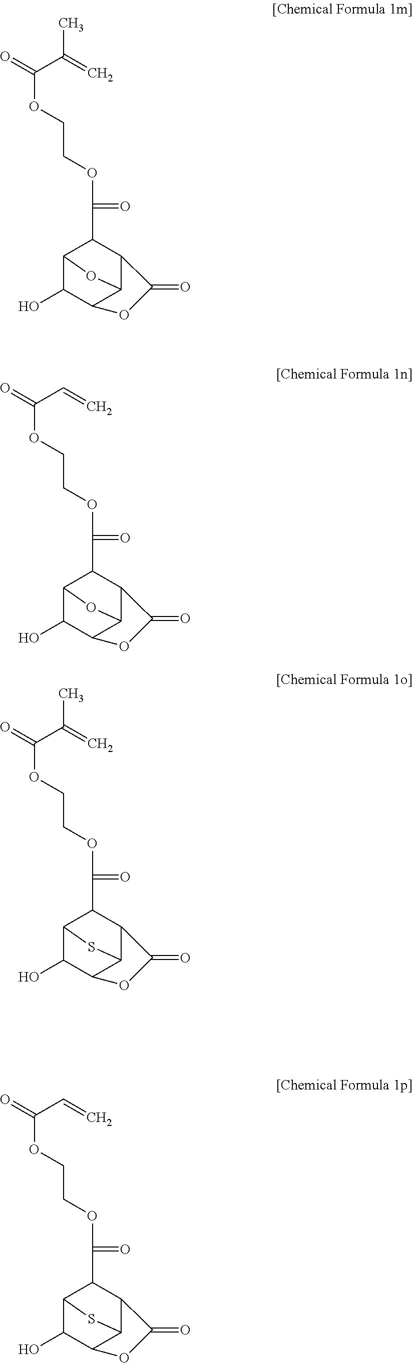

wherein, Me is a methyl group, Et is an ethyl group, iPr is an isopropyl group, and tBu is a t-butyl group.

The lactone-group-containing (meth)acrylate compound represented by Chemical Formula 1 may be usefully applied as, e.g., a repeating unit of a photosensitive polymer. The new lactone group may improve sensitivity, resolution, and etching resistance through a hydrogen bond with an acid. Also, the photosensitive polymer may overcome limitations on sensitivity, resolution, and etching resistance in an ArF resist material, and therefore may be usefully applied to a semiconductor device requiring higher resolution.

The lactone-group-containing (meth)acrylate compound represented by the above Chemical Formula 1 may be prepared through the following process.

A maleic anhydride derivative and one of a cyclopentadiene derivative, a cyclo-hexadiene derivative, a furan derivative, or a thiophene derivative may be reacted, e.g., through a Diels-Alder reaction, to prepare a 5-norbornene-2,3-dicarboxylic acid anhydride derivative represented by the following Chemical Formula A. A method of preparation of the compound represented by the following Chemical Formula A is generally well-known, and a detailed description thereof is not considered necessary herein.

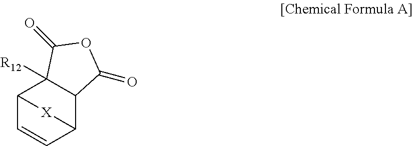

wherein, R₁₂ is hydrogen, or a linear, branched, or cyclic substituted or unsubstituted alkyl group, for example a methyl group, an ethyl group, a propyl group, an isopropyl group, an n-butyl group, a sec-butyl group, a tert-butyl group, a tert-amyl group, an n-pentyl group, an n-hexyl group, a cyclopentyl group, a cyclohexyl group, a cyclopentylmethyl group, a cyclopentylethyl group, a cyclohexylmethyl group, or a cyclohexylethyl group, and X is (CH₂)ₙ (n is 1 or 2), O, or S.

The compound represented by the above Chemical Formula A may be reacted with peroxide to prepare an epoxy compound represented by the following Chemical Formula B.

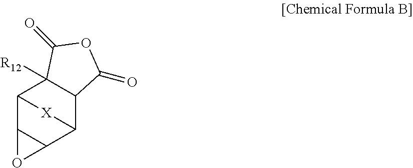

As used herein, the term “peroxide” broadly refers to a peroxide without a substituent, hydroperoxide or salts thereof, permanganic acid or salts thereof, organic peroxide, inorganic peroxide, and the like.

The compound represented by the above Chemical Formula A and the peroxide may be reacted through the following two processes.

In the first method, the reaction may be performed using an inorganic peroxide in the presence of a metal compound.

Examples of the metal included in the metal compound include W, Mo, V, Mn, or a combination thereof, but are not limited thereto. Examples of the metal compound include any compound including the above metal, without limitation. Examples of the metal compound include oxides, oxo acids including metal or salts thereof, sulfides, halides, oxy halides, borides, carbides, nitrides, peroxides, organometallic compounds, and the like, and it may be used singularly or in a combination.

Examples of the oxides include tungsten oxide, molybdenum oxide, vanadium oxide, manganese oxide, and the like.

Examples of the oxo acids include metallic acids such as tungstic acid, molybdic acid, vanadic acid, and manganic acid; isopoly acids such as isopoly tungstic acid, isopoly molybdic acid, and isopoly vanadic acid; and hetero polyacids including a first metal element including W, Mo, V, Mn, or a combination thereof, and a second metal element other than the first metal element. Examples of the second metal element include phosphorus or silicon, and in an embodiment, phosphorus is preferable.

Examples of salts of the oxo acids include an alkaline metal salts such as a sodium salt, a potassium salt, and the like, and an alkaline earth salt such as a magnesium salt, a calcium salt, a barium salt, and the like.

Examples of the peroxides include peroxide, hydrogen peroxide, permanganic acid, permanganate, and the like. Pure hydrogen peroxide may be used for the hydrogen peroxide, but considering the advantages of easy handling, hydrogen peroxide diluted with a suitable solvent such as water may be used. The amount of the hydrogen peroxide used may be, e.g., 0.9 to 5 moles based on 1 mole of a 5-norbornene-2,3-dicarboxylic acid anhydride derivative, and in an embodiment, it may be 1.0 to 2.0 moles.

The amount of the metal compound used may be, e.g., 0.0001 to 2 moles based on 1 mole of a 5-norbornene-2,3-dicarboxylic acid anhydride derivative, and in an embodiment, it may be 0.0005 to 0.5 moles.

In a second method, a reaction between the compound represented by the above Chemical Formula A and peroxide may be performed using organic peroxide as the peroxide.

Examples of the organic peroxide include performic acid, peracetic acid, m-chloroperbenzoic acid, and the like, and it may be used singularly or in a combination. The amount of the organic peroxide used may be, e.g., 0.8 to 5 moles based on 1 mole of a 5-norbornene-2,3-dicarboxylic acid anhydride derivative, and in an embodiment, it may be about 1.5 moles.

Because the second method of reacting the compound represented by Chemical Formula A and organic peroxide provides high selectivity, it does not produce side-reactions or by-products that are caused by complex purification processes, and does not need a post-treatment process after the reaction due to non-uniform reaction, the second method is usefully performed, but according to the compound the method may be selected without limitation.

The compounds represented by the above Chemical Formula B and the following Chemical Formula C may be reacted to prepare the compound represented by the above Chemical Formula 1.

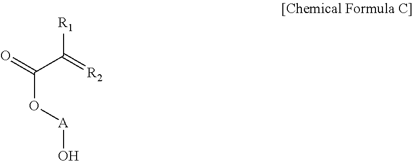

In the above Chemical Formula C, R₁, R₂, and A are the same as defined in Chemical Formula 1. Chemical Formula C may be used to introduce a (meth)acrylate moiety into the resultant polymer. It will be appreciated that the term “(meth)acrylate,” as used herein, is not limited to R₁ being hydrogen or a methyl group, and encompasses implementations wherein R₁ is hydrogen, a methyl group, or CH₂CO₂R₅ as defined in Chemical Formula 1.

The preparing method for the following Chemical Formula C is generally well-known, and a detailed description thereof is not considered necessary herein.

The reaction with the compound represented by Chemical Formula B and the compound represented by Chemical Formula C may be performed using a solvent or without a solvent. Examples of the solvent include alcohols such as t-butylalcohol; halogenated hydrocarbons such as chloroform, dichloromethane, and the like; aromatic hydrocarbons such as benzene; aliphatic hydrocarbons such as octane; cyclic hydrocarbons such as cyclohexane; amides such as N,N-dimethyl formamide; nitriles such as acetonitrile; ethers such as ethyl ether, tetrahydrofuran, and the like; and esters such as ethyl acetate, acetic acid, organic acid, and water. The solvents may be used singularly or in a combination.

The temperature during the reaction may generally range from about 0 to about 150° C., and in an embodiment, may range from about 50 to about 100° C.

Further, a basic compound, e.g., an amine base such as triethylamine or pyridine, and the like may be used to increase reaction rate and yield. The amount of the basic compound used may range from, e.g., 0.001 to 2 moles based on 1 mole of the compound represented by Chemical Formula C, and in an embodiment, it may range from 0.05 to 1.2 moles.

In another embodiment, a photosensitive polymer, including a repeating unit derived from the aromatic (meth)acrylate represented by Chemical Formula 1, and a repeating unit derived from the compound represented by the following Chemical Formula 2, is provided. The photosensitive polymer may be a terpolymer including a repeating unit derived from the compound represented by the following Chemical Formula 3, and in an embodiment, it may be a quaternary copolymer further including a repeating unit derived from the compound represented by the following Chemical Formula 4.

The photosensitive polymer may be any copolymer without limitation for the kind and number of a repeating unit, for example, a block copolymer including regularly repeated repeating units or a random copolymer including randomly repeated repeating units.

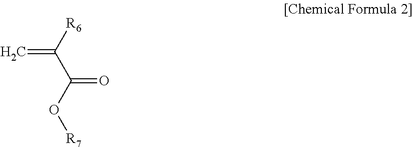

wherein, R₆ is hydrogen or a methyl group,

R₇ is a C₄ to C₂₀ acid-labile group being decomposed under an acid catalyst, for example an alkyl group, a norbornyl group, an isobornyl group, a cyclodecanyl group, a norbornyl having a lower alkyl substituent, an isobornyl having a lower alkyl substituent, a cyclodecanyl group having a lower alkyl substituent, an adamantyl group having a lower alkyl substituent, alkoxycarbonyl, alkoxycarbonyl alkyl, amyloxycarbonyl, amyloxycarbonyl alkyl, 2-tetrahydropyranyloxycarbonyl alkyl, 2-tetrahydrofuranyloxycarbonyl alkyl, a tertiary alkyl group, or an acetal group.

More specific examples of R₇ include 2-methyl-2-norbornyl, 2-ethyl-2-norbornyl, 2-methyl-2-isobornyl, 2-ethyl-2-isobornyl, 8-methyl-8-tricyclodecanyl, 8-ethyl-8-tricyclodecanyl, 2-methyl-2-adamantyl, 2-ethyl-2-adamantyl, 2-propyl-2-adamantyl, t-butoxycarbonyl, t-butoxycarbonylmethyl, t-amyloxycarbonyl, t-amyloxycarbonylmethyl, 1-ethoxyethoxycarbonylmethyl, 2-tetrahydropyranyloxycarbonyl alkyl, 2-tetrahydrofuranyloxycarbonyl alkyl, alkoxycarbonyl alkyl, amyloxycarbonyl, amyloxycarbonyl alkyl, 2-tetrahydropyranyloxycarbonyl alkyl, 2-tetrahydrofuranyloxycarbonylalkyl, triethylcarbyl, an 1-ethylcyclopentyl group, t-amyl group, and an acetal group.

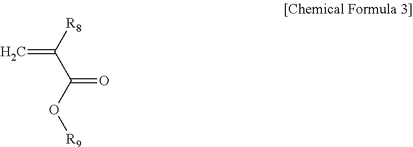

wherein, R₈ is hydrogen or a methyl group,

R₉ is hydrogen, or an alkyl group or cycloalkyl group, examples of the alkyl group and the cycloalkyl group including a hydroxy group, a carboxyl group, and a combination thereof, for example 2-hydroxyethyl, 3-hydroxy-1-adamantyl, and the like.

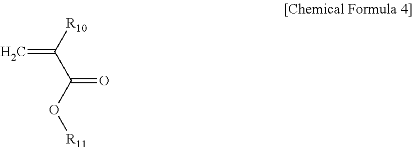

wherein, R₁₀ is hydrogen or a methyl group, and

R₁₁ is a lactone-derived group, and in an embodiment is a substituent having a structure represented by the following Chemical Formula 5 or 6.

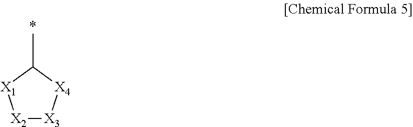

wherein, in the above Chemical Formula 5, two adjacent groups of X₁ to X₄ are CO and O, and a remaining two of X₁ to X₄ except for CO and O are CR″ (where R″ is hydrogen, a C₁ to C₄ alkyl, or an alkylene forming a fused ring with the five-member ring).

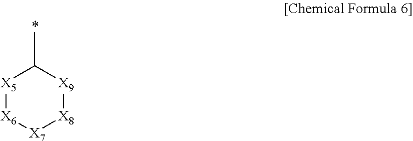

wherein, in the above Chemical Formula 6, two adjacent groups of X₅ to X₉ are CO and O, and a remaining three of X₅ to X₉ except for CO and O are CR″ (where R″ is hydrogen, a C1 to C4 alkyl, or an alkylene forming a fused ring with the six-member ring); or one of X₅ to X₉ is CR′″ (where R′″ is hydrogen, a C₁ to C₄ alkyl, an ester-containing alkylene forming a fused ring with the six-member ring), and at least two of R′″ are linked to each other to from a lactone ring.

In an embodiment, R₁₁ may be butyrolactonyl, valerolactonyl, 1,3-cyclohexanecarbolactonyl, 2,6-norbornanecarbolacton-5-yl, or 7-oxa-2,6-norbornanecarbolacton-5-yl.

According to an embodiment, the photosensitive polymer as described above may be a copolymer including repeating units represented by Chemical Formulae 1 and 2, it may be a terpolymer including repeating units derived from the compound represented by Chemical Formulae 1, 2, and 3, or it may be a quaternary copolymer including repeating units derived from the compound represented by Chemical Formulae 1, 2, 3, and 4.

Herein, when the photosensitive polymer is a copolymer, and the mole fraction of the repeating unit derived from the compound represented by the above Chemical Formula 1 is a, and the mole fraction of the repeating unit derived from the compound represented by the above Chemical Formula 2 is b, a and b may be defined as follows.

a/(a+b)=0.2 to 0.8 a

b/(a+b)=0.2 to 0.8 b

Herein, when the photosensitive polymer is a terpolymer, and the mole fraction of the repeating unit derived from the compound represented by Chemical Formula 1 is c, the mole fraction of the repeating unit derived from the compound represented by Chemical Formula 2 is d, and the mole fraction of the repeating unit derived from the compound represented by Chemical Formula 3 is e, c, d, and e may be defined as follows.

c/(c+d+e)=0.3 to 0.7, c

d/(c+d+e)=0.2 to 0.8, d

and

e/(c+d+e)=0.1 to 0.4 e

Also, when the photosensitive polymer is a quaternary copolymer, and the mole fraction of the repeating unit derived from the compound represented by Chemical Formula 1 is s, the mole fraction of the repeating unit derived from the compound represented by Chemical Formula 2 is p, the mole fraction of the repeating unit derived from the compound represented by Chemical Formula 3 is q, and the mole fraction of the repeating unit derived from the compound represented by the above Chemical Formula 4 is r, s, p, q, and r may be defined as follows.

In an embodiment, p/(p+q+r+s)=0.2 to 0.5, q/(p+q+r+s)=0 to 0.5, r/(p+q+r+s)=0.1 to 0.4, and s/(p+q+r+s)=0.01 to 0.2. In another embodiment, q/(p+q+r+s)=0.3 to 0.5, r/(p+q+r+s)=0.1 to 0.4, and s/(p+q+r+s)=0.1 to 0.2.

The photosensitive polymer may have a weight average molecular weight (Mw) of about 3,000 to about 20,000.

When the photosensitive polymer has polydispersity (Mw/Mn) ranging from about 1.5 to about 2.5, it may have excellent etching resistance and resolution.

The photosensitive polymers according to an embodiment are copolymers obtained from compounds including a lactone group provided by Chemical Formula 1. The photosensitive polymers may provide a resist composition having excellent sensitivity, resolution, and etching resistance. When the resultant resist composition is applied to a photolithographic process, it may provide excellent lithography performance.

According to another embodiment, a resist composition including the photosensitive polymer is provided.

The resist composition may include (a) the photosensitive polymer, (b) a photoacid generator (PAG), and (c) a solvent.

Hereinafter, the components of the resist composition according to an embodiment are described in more detail.

(a) Photosensitive Polymer

The photosensitive polymer is the same as described above.

The photosensitive polymer may be included in an amount of about 5 to about 15 parts by weight based on 100 parts by weight of the resist composition. When the photosensitive polymer is included in the above range, it can provide the resist composition with excellent sensitivity, resolution, and etching resistance characteristics.

(b) Photoacid Generator (PAG)

The photoacid generator may be an inorganic onium salt, an organic sulfonate, a mixture thereof, etc. Examples of the photoacid generator include a sulfonate or iodonium salt such as a triarylsulfonium salt, a diaryl iodonium salt, a sulfonate, or mixtures thereof. In an embodiment, the photoacid generator may include triarylsulfonium triflate, diaryliodonium triflate, triarylsulfonium nonaflate, diaryliodonium nonaflate, succinimidyl triflate, 2,6-dinitrobenzyl sulfonate, or mixtures thereof.

The photoacid generator may be added in an amount of about 1 to about 15 parts by weight based on 100 parts by weight of the photosensitive polymer. When the photoacid generator is added within the range, the exposure dose with respect to the resist composition may be controlled, and the transmission of the resist composition may also be controlled.

(c) Solvent

The solvent may include, e.g., one or more of propylene glycol monomethyl ether acetate (PGMEA), propylene glycol methyl ether (PGME), ethyl lactate (EL), cyclohexanone, 2-heptanone, and so on.

The solvent may be added as the balance amount of the resist composition without limitation. In an embodiment, the solvent may be added in an amount of about 80 parts by weight to about 95 parts by weight based on 100 parts by weight of the resist composition.

(d) Additive

The resist composition may further include an organic base as a quencher in order to control the exposure dose and to form a resist profile.

The organic base may be, e.g., an amine-based compound such as triethylamine, triisobutylamine, trioctylamine, triisodecylamine, triethanolamine, or mixtures thereof.

In an embodiment, the amount of organic base ranges from about 0.1 to about 1 part by weight based on 100 parts by weight of the photosensitive polymer. When the organic base is added within the range, the exposure dose is not increased excessively, but a desirable effect may be obtained and the pattern is well formed.

The process to form a desirable pattern with the resist composition obtained from the above process may be as follows.

A bare silicon wafer or a silicon wafer including an underlayer such as a silicon oxide layer, a silicon nitride layer, or a silicon nitride oxide layer on the upper surface may be treated with HMDS (hexamethyldisilazane) or an organic anti-reflection coating (bottom anti-reflective coating). Thereafter, the resist composition may be coated on the silicon wafer at a thickness of about 100 to about 150 nm to provide a resist layer.

The silicon wafer formed with the resist layer may be soft-baked (SB, the same as pre-baking) at a temperature of about 90 to about 120° C. for about 60 to about 90 seconds to remove the solvent, and may be exposed to an exposure light source, for example, ArF or EUV (extreme UV), E-beam, and so on. In order to perform chemical reaction in the exposed region of the resist layer, it may be subjected to PEB (post-exposure baking) at a temperature of about 90 to about 120° C. for about 60 to about 90 seconds.

Then, the resist layer may be developed in, e.g., a basic aqueous developing solution. A region, e.g., the exposed region, may have very high solubility to the basic aqueous developing solution, and so may be easily dissolved and removed during the development. Tetramethyl ammonium hydroxide (TMAH) may be used as the basic aqueous developing solution. When the exposure light source used is an ArF excimer laser, an 80 to 100 nm line and space pattern may be obtained at a dose of about 5 to about 50 mJ/cm².

The resist pattern obtained from the above process may be used as a mask, and the underlayer (such as a silicon oxide layer) may be etched by using an etching gas, e.g., a plasma of a halogen gas or a fluorocarbon gas such as CF₄. The resist pattern that remains on the wafer may be removed by using a stripper to provide a desired pattern, e.g., a silicon oxide layer pattern.

The following Examples and Comparative Examples are provided in order to set forth particular details of one or more embodiments. However, it will be understood that the embodiments are not limited to the particular details described. Further, the Comparative Examples are set forth to highlight certain characteristics of certain embodiments, and are not to be construed as either limiting the scope of the invention as exemplified in the Examples or as necessarily being outside the scope of the invention in every respect.

### Example 1-1

### Synthesis of Monomer (Chemical Formula 1a)

As shown in the following Reaction Scheme 1, a monomer (Chemical Formula 1a) was synthesized.

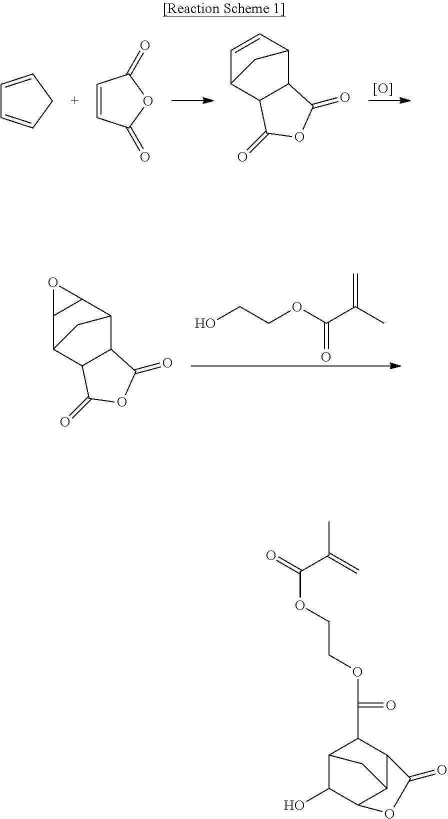

1^{st} step: 324.3 g of maleic anhydride was dissolved in 300 ml of benzene. Then, 327.2 g of cyclopentadiene was dripped into the solution at 40° C. or below for 2 hours. After dripping, the obtained mixture was agitated at room temperature for 12 hours and concentrated under a reduced pressure such that 508.5 g of 5-norbornene-2,3-dicarboxylic acid anhydride (I) was prepared. Herein, yield was 93.7%.

2^{nd} step: 508.5 g of the prepared 5-norbornene-2,3-dicarboxylic acid anhydride (I) and 50.8 g of hydrogen peroxide were dissolved in 2 L of dimethyl formamide. The solution was reacted [O] at 50° C. for 12 hours, and cooled to room temperature. The cooled product was filtered and dried under a reduced pressure such that 406 g of compound (II) was prepared.

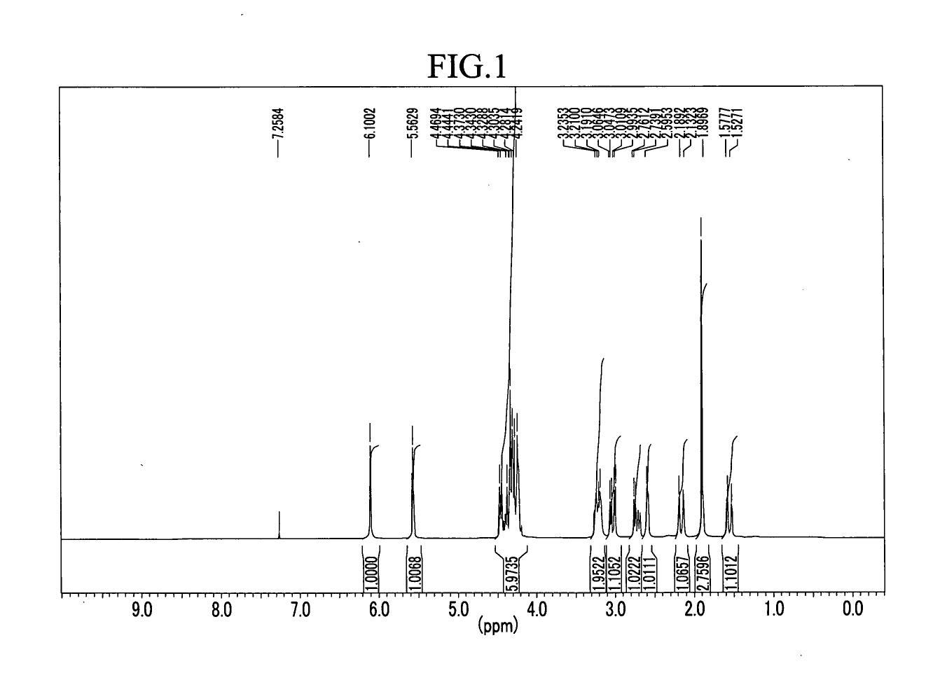

3^{rd} step: 406 g of the compound (II) was dissolved in 5 L of ethyl acetate, and 300 g of 2-hydroxyethylmethacrylate was added to the solution. The obtained mixture was heated at 70° C. for 18 hours, and cooled to room temperature. The cooled product was extracted using 5 L of water 3 times, and an organic phase was dried and then concentrated under a reduced pressure. The obtained reactant was purified with column chromatography (hexane:ethyl acetate=2:1) such that 407 g of monomer (Chemical Formula 1a) according to an embodiment was obtained. Herein, yield was 58.2%. NMR of the prepared monomer (Chemical Formula 1a) was measured, and the results are shown in FIG. 1.

### Example 1-2

### Synthesis of Monomer (Chemical Formula 1 m)

Furan was used instead of cyclopentadiene to obtain a monomer having a structure represented by the following Chemical Formula 1m (3^{rd} step yield: 48%).

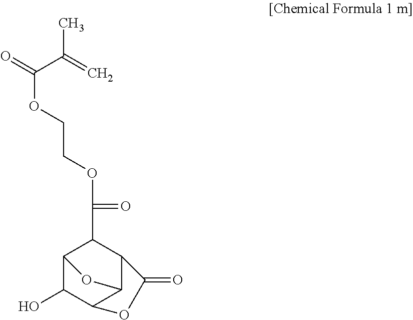

### Example 2-1

### Synthesis of Photosensitive Polymer

5 mmol of the monomer (Chemical Formula 1a) synthesized according to Example 1-1, 35 mmol of γ-butyrolactonyl methacrylate (GBLMA), 35 mmol of 2-methyl-2-adamantyl methacrylate (MAMA), and 25 mmol of 4-hydroxy-2-adamantyl methacrylate (HAMA) were put in a flask, and dissolved in a dioxane solvent of twice the total amount of the monomer. Then, 10 mmol of dimethyl-2,2′-azobis(2-methylpropionate) (V601, Wako Pure Chemical Industries, Ltd.) was added to the solution as a polymerization initiator, and the mixture solution was polymerized at 80° C. for about 4 hours.

When the polymerization was complete, the reactants were slowly precipitated in an excess of a diethylether solvent, and then the precipitate was filtered. The filtered precipitate was dissolved in a predetermined amount of tetrahydrofuran, and then reprecipitated in diethylether. Next, the precipitate was dried in a 50° C. vacuum oven for 24 hours such that a photosensitive polymer including repeating units according to the following Chemical Formulae 8a, 8b, 8c, and 8d was obtained (yield: 53%). Herein, the photosensitive polymer had a weight average molecular weight (Mw) of 7,900 and polydispersity (Mw/Mn) of 1.9. The mole fraction (p:q:r:s) of the following Chemical Formulae 8a, 8b, 8c, and 8d was 0.35:0.35:0.25:0.05.

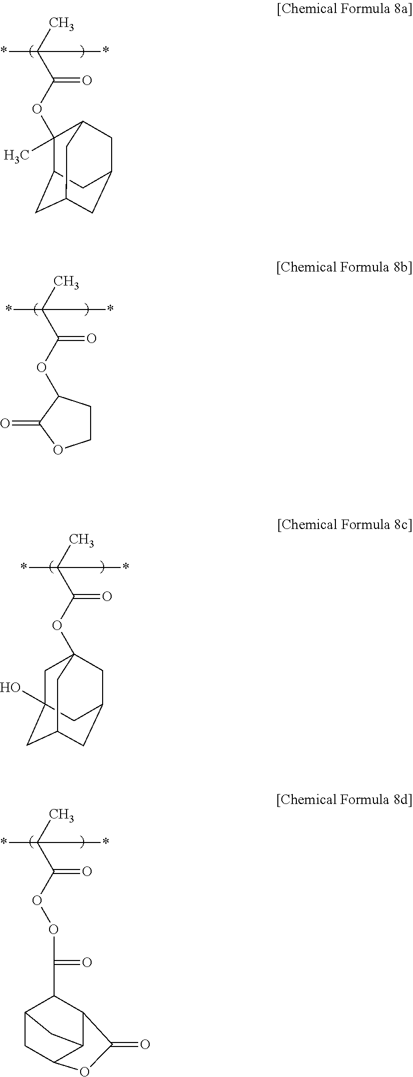

### Example 2-2

### Synthesis of Photosensitive Polymer

5 mmol of the monomer (Chemical Formula 1m) synthesized according to the Example 1-2, 35 mmol of γ-butyrolactonyl methacrylate (GBLMA), 35 mmol of 2-methyl-2-adamantyl methacrylate (MAMA), and 25 mmol 4-hydroxy-2-adamantyl methacrylate (HAMA) were put in a round flask, and dissolved in a dioxane solvent of three times the total amount of the monomer. Then, 10 mmol of dimethyl-2,2′-azobis (2-methylpropionate) (V601, Wako Pure Chemical Industries, Ltd.) was added to the solution as a polymerization initiator, and the mixture solution was polymerized at 80° C. for about 4 hours.

When the polymerization was complete, polymerization was performed with the same method as in Example 2-1 to obtain a photosensitive polymer including repeating units of Chemical Formulae 8a, 8b, 8c, and 9d (yield: 60%). Herein, the photosensitive polymer has a weight average molecular weight (Mw) of 8,600 and polydispersity (Mw/Mn) of 1.6. The mole fraction (p:q:r:s) of the following Chemical Formulae 8a, 8b, 8c and 9d was 0.35:0.35:0.25:0.05.

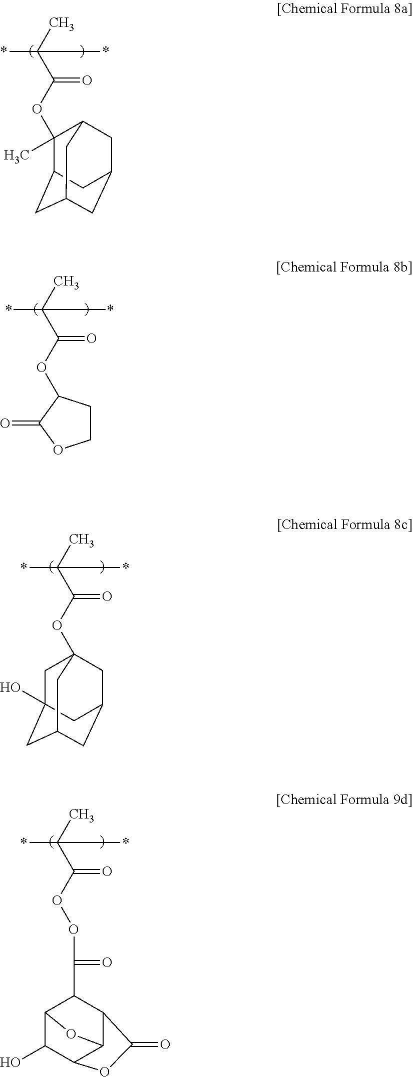

### Example 2-3

### Synthesis of Photosensitive Polymer

10 mmol of the monomer (Chemical Formula 1 m) synthesized according to the Example 1-2, 35 mmol of γ-butyrolactonyl methacrylate (GBLMA), 30 mmol of 2-methyl-2-adamantyl methacrylate (MAMA), and 25 mmol of 4-hydroxy-2-adamantyl methacrylate (HAMA) were put in a round flask, and dissolved in a dioxane solvent of three times the total amount of the monomer. Then, 10 mmol of dimethyl-2,2′-azobis(2-methylpropionate) (V601, Wako Pure Chemical Industries, Ltd.) was added to the solution as a polymerization initiator, and the mixture solution was polymerized at 80° C. for about 4 hours.

When the polymerization was complete, precipitation was performed with the same method as in the Example 2-1 to obtain a photosensitive polymer including repeating units of Chemical Formulae 8a, 8b, 8c, and 9d (yield: 58%). Herein, the photosensitive polymer had a weight average molecular weight (Mw) of 8,100 and polydispersity (Mw/Mn) of 1.7.

The mole fraction (p:q:r:s) of the following Chemical Formulae 8a, 8b, 8c and 9d was 0.30:0.35:0.25:0.05.

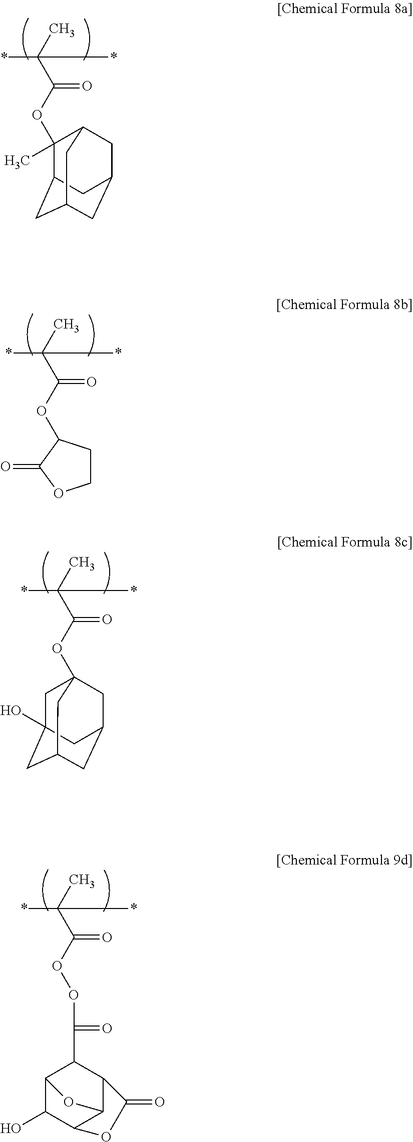

### Examples 3-1 to 3-3

### Preparation of Resist Composition and Lithography Performance

For each resist composition, 0.8 g of the respective photosensitive polymer synthesized according to Examples 2-1 to 2-3 was dissolved in 17 g of polyethylene glycol methacrylate/ethyl lactate (6/4 volume ratio) solvent with 0.02 g of a triphenylsulfonium nonaflate (TPS) photoacid generator, and then 1 mg of triethanol amine as an organic base was completely dissolved in the solution to prepare respective resist compositions.

The prepared resist composition was filtered using a 0.1 μm membrane filter. The filtered resist composition was spin coated to be 0.5 μm thick on a silicon wafer. Then, it was soft-baked (SB: pre-baking) at a temperature of 110° C. for 60 seconds.

It was exposed to light using an ArF scanner (0.78 NA, dipole), post-exposure baked (PEB), and then developed in a 2.38 wt % tetramethylammonium hydroxide aqueous solution for 60 seconds.

Resist Resolution Evaluation

The prepared resist compositions according to Examples 3-1 to 3-3 were evaluated as follows.

First, sensitivity (threshold energy, Eₜₕ, mJ/cm²) was measured.

The optimum exposure dose (Eₒₚ, mJ/cm²) was determined when a 0.25 μm line and space pattern was formed in a line width ratio of 1 to 1, and the resist resolution was evaluated as the minimum line width (microns, m) of the exposure dose.

The resolution of the resist pattern was measured using a scanning electron microscope.

The results are shown in the following Table 1.

TABLE 1

| | Photosensitive | Sensitivity | Resolution |
|---|---|---|---|
| Example | polymer | (mJ/cm²) | (nm) |
| 3-1 | Example 2-1 | 6.3 | 0.20 |
| 3-2 | Example 2-2 | 6.4 | 0.18 |
| 3-3 | Example 32-3 | 6.4 | 0.15 |

Resist Etching Rate Evaluation

Each of the photosensitive polymers synthesized according to Examples 2-1 to 2-3 and a comparative polymer (polymethylmethacrylic acid, weight average molecular weight: 10,000) were dissolved in cyclohexanone, and then they were respectively spin coated to a 1.0 μm thickness on a silicon wafer. Then, they were pre-baked (soft baking: SB) at a temperature of 110° C. for 60 seconds. The photosensitive polymers were evaluated regarding etching rate (Å/min) while etching by a chlorine-based gas and a fluorine-based gas. The evaluation results are shown in the following Table 2.

TABLE 2

| Example | Chlorine-based gas | Fluorine-based gas |
|---|---|---|
| 2-1 | 1,700 | 1,650 |
| 2-2 | 1,740 | 1,680 |
| 2-3 | 1,720 | 1,670 |
| Comparison | 2,500 | 2,250 |

From the results in Tables 1 and 2, it can be seen that the photosensitive polymers according to Examples 2-1 to 2-3 exhibited excellent resolution and etching resistance.

A general ArF resist may include an acryl-based or methacryl-based polymer, e.g., a poly(methacrylate)-based polymer. However, such polymers may present difficulties as a result of poor sensitivity from acid diffusion, poor resolution, and/or difficulties during etching. A lactone pendant group may be introduced into a polymer, as the lactone group may suppress acid diffusion through a hydrogen bond with an acid. However, even then, a polymer having a lactone group has a limit for sensitivity, resolution, and etching resistance, characteristics that are required for a photosensitive resin.

In contrast, embodiments may provide a lactone-group-containing (meth)acrylate compound having a low manufacturing cost and being suitable for preparation of a photosensitive polymer. Embodiments may also provide a photosensitive polymer including a repeating unit derived from the lactone-group-containing (meth)acrylate compound, and providing good hydrogen bonding capability to control acid diffusion resulting in improvement of sensitivity, resolution, and etching resistance in a lithographic process. The photosensitive polymer may be applied to a chemically-amplified resist composition. Embodiments may provide a resist composition including the photosensitive polymer and providing excellent lithography performance in a lithographic process using a light source emitting light in an ultrashort wavelength region such as a 193 nm region or EUV (13.5 nm). The resist composition including the photosensitive polymer has improved sensitivity, resolution, and etching resistance during manufacture of a semiconductor device, as compared with a general ArF resist. Therefore, embodiments may be useful to fabricate a next generation semiconductor.

Example embodiments have been disclosed herein, and although specific terms are employed, they are used and are to be interpreted in a generic and descriptive sense only and not for purpose of limitation. Accordingly, it will be understood by those of skill in the art that various changes in form and details may be made without departing from the spirit and scope of the present invention as set forth in the following claims.

What is claimed is:

1. A lactone-group-containing (meth)acrylate compound represented by the following Chemical Formula 1, 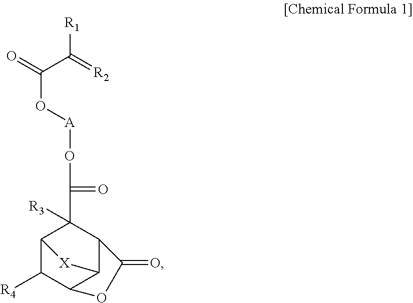
wherein, in the above Chemical Formula 1, R₁ is hydrogen, a methyl group, or CH₂CO₂R₅,
R₂ is CR′R″ where R′ and R″ are independently hydrogen, a methyl group, or CO₂R₅,
R₃ is hydrogen, or a linear, branched, or cyclic substituted or unsubstituted alkyl group,
R₄ is a hydroxy or CO₂R₅,
X is (CH₂)ₙ (where n is 1 or 2), O, or S,
R₅ is a linear, branched, or cyclic substituted or unsubstituted alkyl group, and
A is a linear, branched, or cyclic substituted or unsubstituted alkylene group.

2. The (meth)acrylate compound as claimed in claim 1, wherein the (meth)acrylate compound is a compound represented by one of the following Chemical Formulae 1a to 1p,

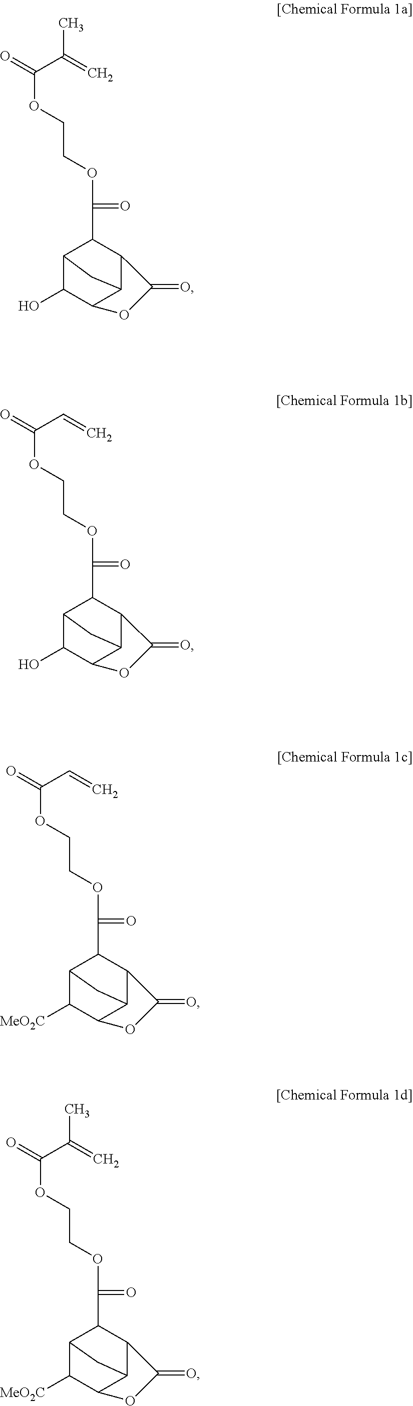

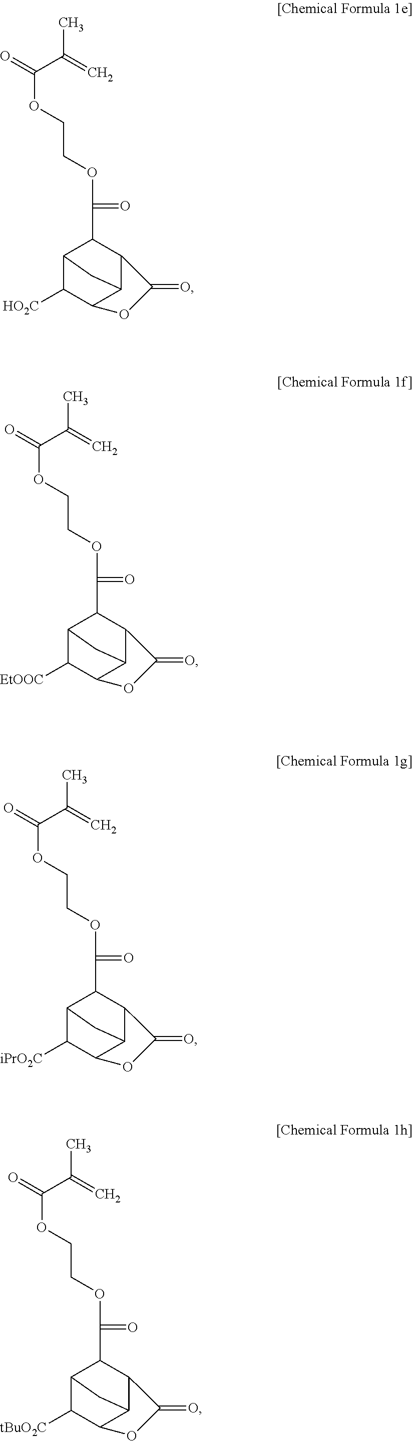

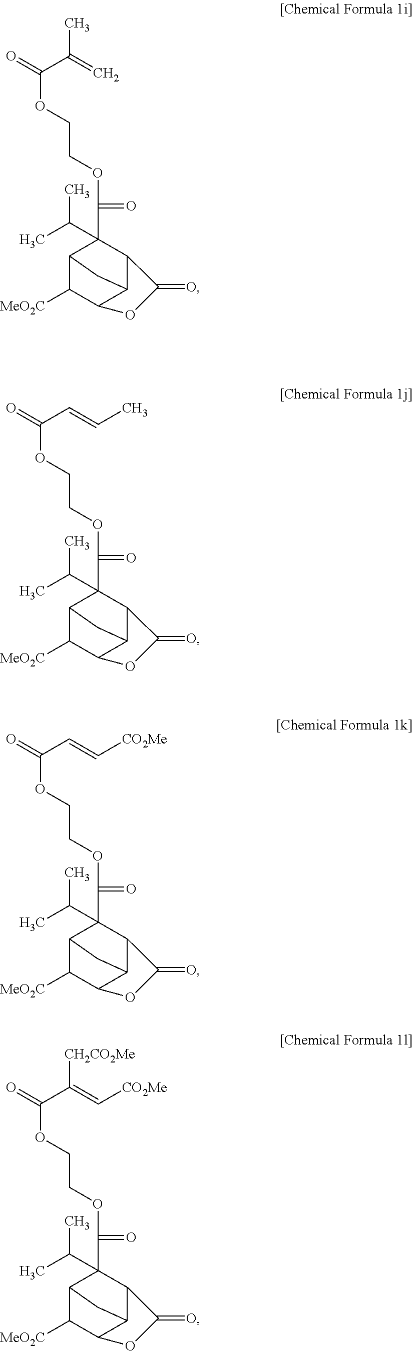

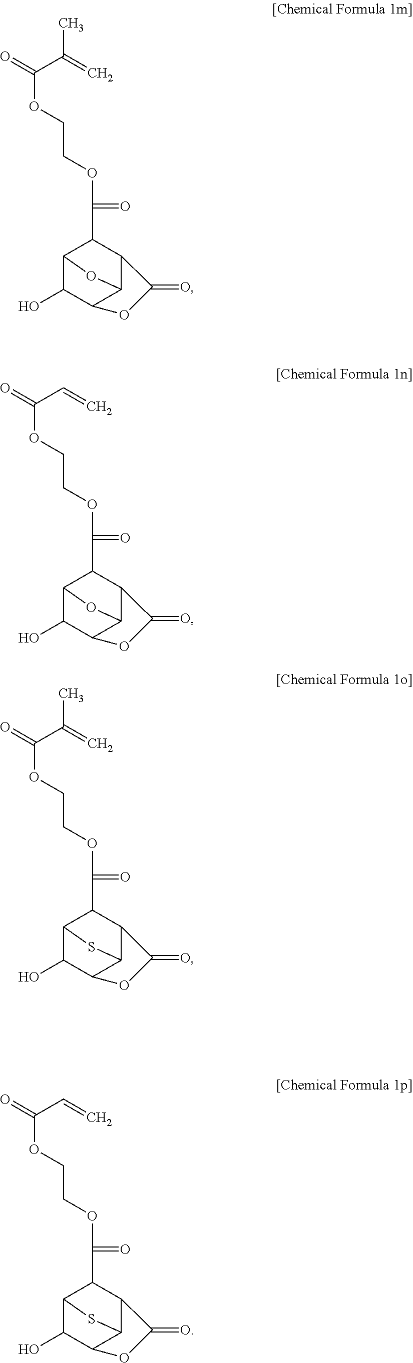

3. A photosensitive polymer, comprising:
a repeating unit derived from a compound represented by the following Chemical Formula 1; and
a repeating unit derived from a compound represented by the following Chemical Formula 2, 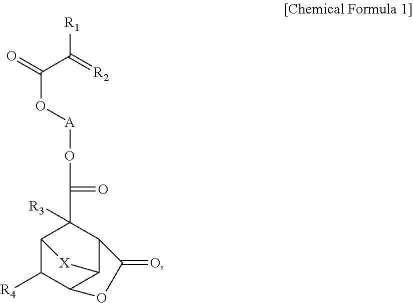
wherein, in the above Chemical Formula 1, R₁ is hydrogen, a methyl group, or CH₂CO₂R₅,
R₂ is CR′R″ where R′ and R″ are independently hydrogen, a methyl group, or CO₂R₅,
R₃ is hydrogen, or a linear, branched, or cyclic substituted or unsubstituted alkyl group,
R₄ is a hydroxy or CO₂R₅,
R₅ is a linear, branched, or cyclic substituted or unsubstituted alkyl group,
X is (CH₂)ₙ (where n is 1 or 2), O, or S, and
A is a linear, branched, or cyclic substituted or unsubstituted alkylene group, 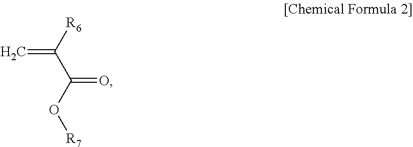
wherein, in the above Chemical Formula 2, R₆ is hydrogen or a methyl group, and
R₇ is a C₄ to C₂₀ acid-labile group being decomposed under an acid catalyst.

4. The photosensitive polymer as claimed in claim 3, wherein R₇ is an alkyl group, a norbornyl group, an isobornyl group, a cyclodecanyl group, a norbornyl having a lower alkyl substituent, an isobornyl having a lower alkyl substituent, a cyclodecanyl group having a lower alkyl substituent, an adamantyl group having a lower alkyl substituent, alkoxycarbonyl, alkoxycarbonyl alkyl, amyloxycarbonyl, amyloxycarbonyl alkyl, 2-tetrahydropyranyloxycarbonyl alkyl, 2-tetrahydrofuranyloxycarbonyl alkyl, a tertiary alkyl group, or an acetal group.

5. The photosensitive polymer as claimed in claim 3, wherein a mole fraction of the repeating unit derived from the compound represented by Chemical Formula 1 ranges from 0.2 to 0.8 and a mole fraction of the repeating unit derived from the compound represented by Chemical Formula 2 ranges from 0.2 to 0.8 based on the total moles of the repeating units derived from the compounds represented by Chemical Formulae 1 and 2.

6. The photosensitive polymer as claimed in claim 3, further comprising:
a repeating unit derived from a compound represented by the following Chemical Formula 3, 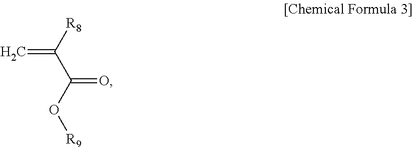
wherein, in the above Chemical Formula 3, R₈ is hydrogen or a methyl group, and
R₉ is hydrogen or an alkyl group or cycloalkyl group including a polar functional group of a hydroxy group, a carboxyl group, or a combination thereof.

7. The photosensitive polymer as claimed in claim 6, wherein R₉ is 2-hydroxyethyl or 3-hydroxy-1-adamantyl.

8. The photosensitive polymer as claimed in claim 6, wherein the photosensitive polymer further comprises a repeating unit derived from a compound represented by the following Chemical Formula 4, 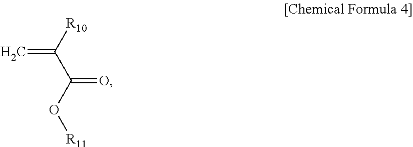
wherein, in the above Chemical Formula 4, R₁₀ is hydrogen or a methyl group, and
R₁₁ is a lactone-derived group.

9. The photosensitive polymer as claimed in claim 8, wherein R₁₁ is a substituent represented by the following Chemical Formula 5 or Chemical Formula 6, 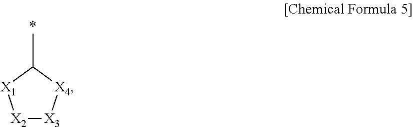
wherein, in the above Chemical Formula 5, two of X₁ to X₄ positioned to be adjacent to each other are CO and O, and the remaining two of X₁ to X₄ except for CO and O are CR″ (where R″ is hydrogen, a C₁ to C₄ alkyl, or an alkylene forming a fused ring with a five-member ring), 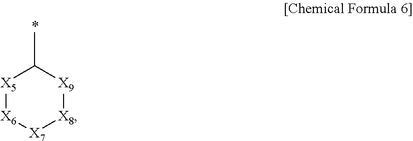
wherein, in the above Chemical Formula 6, two of X₅ to X₉ positioned to be adjacent to each other are CO and O, and the remaining three of X₅ to X₉ except for CO and O are CR″(where R″ is hydrogen, a C₁ to C₄ alkyl, or an alkylene forming a fused ring with a six-member ring); or X₅ to X₉ are CR′″ (where R′″ is hydrogen, a C₁ to C₄ alkyl, an ester-containing alkylene forming a fused ring with the six-member ring), and at least two of R′″ are linked to each other to from a lactone ring.

10. The photosensitive polymer as claimed in claim 3, wherein the photosensitive polymer has a weight average molecular weight (Mw) of about 3,000 to about 20,000.

11. The photosensitive polymer as claimed in claim 3, wherein the photosensitive polymer has a polydispersity (Mw/Mn) of about 1.5 to about 2.5.

12. A resist composition, comprising:
a photosensitive polymer including a repeating unit derived from a compound represented by the following Chemical Formula 1 and a repeating unit derived from a compound represented by the following Chemical Formula 2;
a photoacid generator (PAG); and
a solvent, 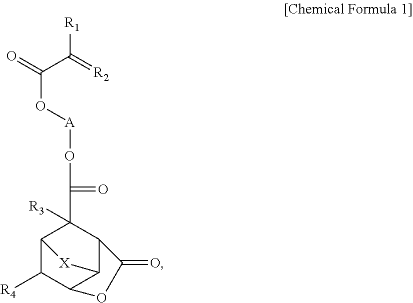
wherein, in the above Chemical Formula 1, R₁ is hydrogen, a methyl group, or CH₂CO₂R₅,
R₂ is CR′R″ where R′ and R″ are independently hydrogen, a methyl group, or CO₂R₅,
R₃ is hydrogen, or a linear, branched, or cyclic substituted or unsubstituted alkyl group,
R₄ is a hydroxy or CO₂R₅,
R₅ is a linear, branched, or cyclic substituted or unsubstituted alkyl group,
X is (CH₂)ₙ (where n is 1 or 2), O, or S, and
A is a linear, branched, or cyclic substituted or unsubstituted alkylene group, 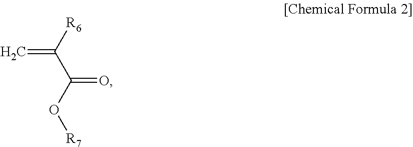
wherein, in the above Chemical Formula 2, R₆ is hydrogen or a methyl group, and
R₇ is a C₄ to C₂₀ acid-labile group being decomposed under an acid catalyst.

13. The resist composition as claimed in claim 12, wherein the photosensitive polymer further includes a repeating unit derived from a compound represented by the following Chemical Formula 3, 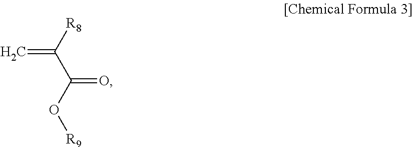
wherein, in the above Chemical Formula 3, R₈ is hydrogen or a methyl group, and
R₉ is hydrogen, an alkyl group, or a cycloalkyl group, the alkyl group and the cycloalkyl group including a hydroxy group, a carboxyl group, or a combination thereof.

14. The resist composition as claimed in claim 13, wherein the photosensitive polymer further includes a repeating unit derived from a compound represented by the following Chemical Formula 4, 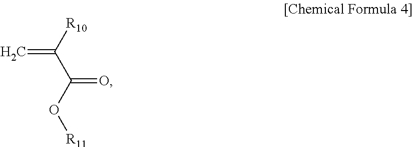
wherein, in the above Chemical Formula 4, R₁₀ is hydrogen or a methyl group, and
R₁₁ is a lactone-derived group.

15. The resist composition as claimed in claim 12, wherein the photosensitive polymer is included in an amount of about 5 to about 15 parts by weight based on 100 parts by weight of a resist composition.

16. The resist composition as claimed in claim 12, wherein the photoacid generator is added in an amount of about 1 to about 15 parts by weight based on 100 parts by weight of the photosensitive polymer.

17. The resist composition as claimed in claim 12, wherein the photoacid generator includes a triarylsulfonium salt, a diaryl iodonium salt, a sulfonate, or a mixture thereof.

18. The resist composition as claimed in claim 12, wherein the organic base is included in an amount of about 0.1 to about 1 part by weight based on 100 parts by weight of the photosensitive polymer.

19. The resist composition as claimed in claim 16, wherein the organic base includes triethylamine, triisobutylamine, trioctylamine, triisodecylamine, triethanolamine, or a mixture thereof.
